SEQUENCE LISTING (2 sequences)

SEQ ID NO: 1 - DNA, 2373 bp, Arabidopsis thaliana
atgtttgaagtcaaaatggggtcaaagatgtgcatgaacgcttcatgtggtacgacttctactgttgaatggaagaaaggttggcctcttcgatctggtcttctcgctgatctctgttatcgttgcggatctgcgtatgagagttctctattctgtgaacaatttcataaggaccaatctggttggagggaatgctatttgtgtagcaagagactacattgtggatgcattgcttctaaggtaacgattgagttaatggactatggtggtgttggttgtagtacatgtgcttgctgccatcaactcaatttgaacacaaggggtgagaatccaggtgtttttagcagattgccaatgaaaacgttagctgataggcaacatgtaaatggcgaaagcggaggaagaaacgaaggcgatctcttttctcagccactagtcatgggcggagataaaagggaagagttcatgcctcaccgtgggtttggtaagctaatgagtccagaaagtacaaccaccgggcataggctggatgctgctggggaaatgcatgaatcatcacctttacagccatctttaaatatgggtttggctgtgaatccgtttagcccatcttttgcaaccgaggctgtcgagggaatgaaacacatcagtccttctcagtccaacatggtccattgctctgcttctaatatactgcaaaagccatcaagacctgctatttcaactcctcctgtggctagtaaatccgctcaggcgcggattggaaggcctcctgtcgaagggcgagggagaggccacttgcttccgcggtattggccaaaatatacggataaagaggttcagcagatctctggaaatttgaatttgaacattgtacctctctttgagaaaactcttagtgccagtgatgctggtcgcattggtcgtctagttcttccaaaagcctgtgcagaggcatattttcctccgattagtcaatccgaaggcattcctttgaaaatccaagatgtgaggggtagggagtggacgttccagttcagatattggcccaataacaatagtagaatgtatgttttagaaggtgtcactccatgcatacagtccatgatgctacaggctggtgatacagtaactttcagtcgggttgatcctggcggaaaactaatcatgggttccaggaaggcagctaatgctggagacatgcagggttgtgggctcaccaacggaacatcaactgaggacacatcatcgtctggtgtaacagaaaacccaccctccataaatggttcctcgtgtatttcactaataccgaaagagttgaatggtatgcctgagaatttgaacagtgagactaacgggggcaggataggtgatgatcctacacgagttaaagagaagaagagaactcgaaccattggtgcaaaaaataagagacttcttttgcatagtgaagaatctatggagctgagactcacttgggaagaagctcaggacttgcttcgtccctctcctagtgtaaagcctaccatcgttgtcattgaggagcaagaaattgaagaatatgacgaacctcctgtctttggaaagaggactatagtcactacaaaaccttcaggtgaacaggaacgatgggcaacttgcgacgactgctctaaatggagaaggttacctgtagatgctcttctttcctttaaatggacatgtatagacaatgtttgggatgtgagtaggtgttcatgttctgcaccggaggagagtctgaaggaacttgagaatgttcttaaagtaggaagagagcacaagaagagaagaactggggaaagccaggcagcaaaaagtcagcaagaaccgtgtggtttggacgcactggcgagtgcagcagtcttaggagacacaataggcgagccagaggtagcgaccacgaccagacatccaaggcacagggctggatgctcttgcatcgtgtgcattcagccaccaagtgggaaaggtaggcacaagcctacatgtggctgcactgtgtgtagcaccgtgaagagaaggttcaagacgcttatgatgaggaggaagaagaagcagttggagcgcgatgtaacagcagcagaagataagaagaagaaggacatggaactggctgagtctgataagagtaaggaggagaaggaagtgaacacagcgagaatagacctgaacagtgatccatacaataaagaagatgttgaagctgttgcggtggagaaagaagagagtcgaaaaagagcaataggacagtgttcgggcgtggtggctcaagacgccagtgatgttttaggagttacagagttagaaggagagggtaagaatgttcgtgaagagccgagagtttcaagctga

SEQ ID NO: 2 - protein, 790 aa, Arabidopsis thaliana
MFEVKMGSKMCMNASCGTTSTVEWKKGWPLRSGLLADLCYRCGSAYESSLFCEQFHKDQSGWRECYLCSKRLHCGCIASKVTIELMDYGGVGCSTCACCHQLNLNTRGENPGVFSRLPMKTLADRQHVNGESGGRNEGDLFSQPLVMGGDKREEFMPHRGFGKLMSPESTTTGHRLDAAGEMHESSPLQPSLNMGLAVNPFSPSFATEAVEGMKHISPSQSNMVHCSASNILQKPSRPAISTPPVASKSAQARIGRPPVEGRGRGHLLPRYWPKYTDKEVQQISGNLNLNIVPLFEKTLSASDAGRIGRLVLPKACAEAYFPPISQSEGIPLKIQDVRGREWTFQFRYWPNNNSRMYVLEGVTPCIQSMMLQAGDTVTFSRVDPGGKLIMGSRKAANAGDMQGCGLTNGTSTEDTSSSGVTENPPSINGSSCISLIPKELNGMPENLNSETNGGRIGDDPTRVKEKKRTRTIGAKNKRLLLHSEESMELRLTWEEAQDLLRPSPSVKPTIVVIEEQEIEEYDEPPVFGKRTIVTTKPSGEQERWATCDDCSKWRRLPVDALLSFKWTCIDNVWDVSRCSCSAPEESLKELENVLKVGREHKKRRTGESQAAKSQQEPCGLDALASAAVLGDTIGEPEVATTTRHPRHRAGCSCIVCIQPPSGKGRHKPTCGCTVCSTVKRRFKTLMMRRKKKQLERDVTAAEDKKKKDMELAESDKSKEEKEVNTARIDLNSDPYNKEDVEAVAVEKEESRKRAIGQCSGVVAQDASDVLGVTELEGEGKNVREEPRVSS

(19) United States
(12) Patent Application Publication
(10) Pub. No.: US 2012/0066794 A1
(43) Pub. Date: Mar. 15, 2012

(54) Increased Seed Oil and Abiotic Stress Tolerance Mediated by HSI2

(76) Inventors: Nirmala Sharma, Saskatoon (CA); Pierre R.J.A. Fobert, Saskatoon (CA)

(21) Appl. No.: 13/320,813

(22) PCT Filed: May 19, 2010
(86) PCT No.: PCT/CA10/00754

§ 371 Date: Nov. 16, 2011

Related U.S. Application Data
(60) Provisional application No. 61/213,314, filed on May 28, 2009.

(51) Int. Cl. C12N 15/82; C12N 5/10; A01H 5/10; C12N 15/63; A01H 5/00

(52) U.S. Cl. 800/281; 435/320.1; 800/298; 435/419; 800/278

(57) ABSTRACT

Increased seed oil content, decreased abscisic acid sensitivity and/or increased drought resistance in a plant may be accomplished by altering expression of a HIGH-LEVEL EXPRESSION OF SUGAR-INDUCIBLE 2 (HSI2) protein in the plant to thereby increase or decrease expression of HSI2 in the plant compared to a plant grown under similar conditions in which the expression of HSI2 was unaltered. Increasing expression of HSI2 increases oil content while decreasing expression of HSI2 decreases abscisic acid sensitivity and/or increases drought resistance.

### CROSS-REFERENCE TO RELATED APPLICATIONS

This application claims the benefit of U.S. Provisional Patent Application U.S. Ser. No. 61/213,314 filed May 28, 2009, the entire contents of which is herein incorporated by reference.

### FIELD OF THE INVENTION

This invention is related to genetic manipulation of plants to alter plant phenotype. In particular, the present invention is related to altering expression of a HIGH-LEVEL EXPRESSION OF SUGAR-INDUCIBLE 2 (HSI2) protein in a plant to alter seed oil content and abiotic stress responses.

### BACKGROUND OF THE INVENTION

In most years, canola is the top Canadian cash crop, generating some $11 B of economic activity. Canola is valued for its superior oil quality and seed oil represents an estimated 80% of the worth of the crop. Recent changes to the registration standards for Canadian canola focus upon an increase in oil content for new varieties and the Canadian industry is targeting a 2.5% increase of seed oil levels to 45% by 2015. Economically, it has been estimated that a 1% increase in seed oil yield translates to an annual value of $80 M CAD. Not surprisingly, increasing seed oil content has been identified by the industry as an important research objective. Achieving this goal is a considerable challenge when one considers that the general trend in the past has been towards a slow upward drift in oil content. According to information from the Canadian Grain Commission, harvest surveys dating back to 1956 show a linear rise (non-significant) of only 0.05% in oil.

Several strategies can be used to increase seed oil content, including conventional breeding, marker assisted breeding and transgenic modifications. Previous and on-going studies using transgenics have focused on manipulation of lipid biosynthetic genes such as diacylglycerol acyltransferase (DGAT) or genes encoding regulatory elements including kinases such as pyruvate dehydrogenase complex kinase (PDCK) and transcription factors such as WRINKLED1 (WRI1). Increases in seed oil modification using the existing approaches are often modest, necessitating the use of multiple genes in combination. Stacking genes with different modes of action may be advantageous, resulting in additive or synergist effects.

HSI2 (HIGH-LEVEL EXPRESSION OF SUGAR-INDUCIBLE GENE 2: AT2G30470), also known as VAL1 (VIVIPAROUS ABA INSENSITIVE3-LIKE), is an Arabidopsis gene that encodes a putative chromatin remodeling factor and transcriptional repressor.

There remains a need in the art for approaches to modifying seed oil content and/or abiotic stress responses in a plant.

### SUMMARY OF THE INVENTION

It has now been found that increasing levels of the HIGH-LEVEL EXPRESSION OF SUGAR-INDUCIBLE 2 (HSI2) protein is an effective means of increasing seed oil content in a plant. In addition, reducing levels of HSI2 in a plant increases tolerance of plants to various abiotic stresses.

Thus, there is provided a method of increasing seed oil content in a plant comprising: introducing into the plant means for encoding a HIGH-LEVEL EXPRESSION OF SUGAR-INDUCIBLE 2 (HSI2) protein to thereby increase expression of HSI2 protein in the plant to thereby increase seed oil content in the plant compared to a plant grown under similar conditions in which the means for encoding the HSI2 protein has not been introduced.

There is further provided a method of decreasing abscisic acid sensitivity and/or increasing drought resistance in a plant comprising: reducing expression of HIGH-LEVEL EXPRESSION OF SUGAR-INDUCIBLE 2 (HSI2) protein in the plant to thereby decrease abscisic acid sensitivity and/or increase drought resistance in the plant compared to a plant grown under similar conditions in which expression of the HSI2 protein has not been reduced.

There is also provided a nucleic acid construct comprising means for encoding a HIGH-LEVEL EXPRESSION OF SUGAR-INDUCIBLE 2 (HSI2) protein operably linked to one or more nucleic acid sequences required for transforming the construct into a cell and/or for expressing or overexpressing the HSI2 protein encoding means in the cell.

There is also provided a cell, seed or plant comprising the nucleic acid construct of the present invention.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts a vector map for a binary T-DNA HSI2 transformation vector.

FIG. 2A depicts a graph showing seed oil content in the hsi2 Arabidopsis thaliana T-DNA insertion mutant line (hsi2-5) and the wild type Col-0.

FIG. 2B depicts a graph showing seed oil content of hsi2 mutants (hsi2-3 and hsi2-5) and three HSI2 complementation lines (complementation in hsi2-5; HSI2 comp-18-3-1, HSI2 comp-12-2-1 and HSI2 comp-19-3-1) compared to wild type (Col-2 and Col-0-1). hsi2-5 is in columbia-0 background and hsi2-3 is in columbia-2 background

FIG. 3A depicts a graph showing germination of hsi2 T-DNA insertion mutant lines and their corresponding wild types on half-strength MS medium supplemented with various concentrations of ABA, 72 hrs after stratification. hsi2-5 compares with the Col-0 wild type and hsi2-3 compares with the Col-2 wild type.

FIG. 3B depicts a graph showing ABI3 and ABI5 gene expression in the absence of ABA 96 hr post-stratification in hsi2-3 seedlings compared to the wild type (Col-2).

FIG. 4A depicts a bar graph showing drought tolerance after 10 days of withholding water for hsi2 T-DNA insertion mutant lines compared to their corresponding wild types. hsi2-5 compares with the Col-0 wild type and hsi2-3 compares with the Col-2 wild type.

FIG. 4B depicts a line graph showing drought tolerance during the period of 14-16 days after withholding water for hsi2-5 compared with the Col-0 wild type. Experiments were repeated three times using different pot sizes, potting mixes and different growth cabinets.

FIG. 4C depicts a line graph showing drought tolerance during the period of 17-19 days after withholding water for hsi2-3 compared with the Col-2 wild type. Experiments were repeated three times using different pot sizes, potting mixes and different growth cabinets.

FIG. 4D depicts a bar graph showing the relative water content after 19 days of withholding water for hsi2 T-DNA insertion mutant lines compared to their corresponding wild types. hsi2-5 compares with the Col-0 wild type and hsi2-3 compares with the Col-2 wild type.

FIG. 4E depicts a graph showing expression of Myb-like transcription factor gene in hsi2 T-DNA insertion mutant lines compared to their corresponding wild types approaching visible wilting. hsi2-5 compares with the Col-0 wild type and hsi2-3 compares with the Col-2 wild type.

### DESCRIPTION OF PREFERRED EMBODIMENTS

An example of one means for encoding a HIGH-LEVEL EXPRESSION OF SUGAR-INDUCIBLE 2 (HSI2) protein is an Arabidopsis nucleic acid molecule (HSI2) (SEQ ID NO: 1). This nucleic acid molecule encodes a putative chromatin remodeling factor and transcriptional repressor protein (SEQ ID NO: 2).

Other means for encoding a HSI2 protein include, for example, nucleic acid molecules that encode proteins having at least 80% sequence identity to SEQ ID NO: 2.

It has now been found that loss of HSI2 reduces the level of seed oil in Arabidopsis thaliana. On the other hand, preliminary oil analysis on primary transformants in Brassica napus overexpressing HSI2 indicate an increase in seed oil content indicating that HSI2 overexpression increases levels of seed oil.

Also, it has now been found that Arabidopsis plants containing T-DNA insertions in HSI2 display greater tolerance to the plant hormone abscisic acid (ABA), which is involved in regulating many physiological processes, including seed maturation and tolerance to abiotic stresses including drought. By subjecting young juvenile plants to drought stress, it is directly shown that hsi2 T-DNA insertion lines are more drought tolerant than their wild type counterparts.

Double mutants in HSI2 and its closest relative in Arabidopsis leads to the production of embryos on seedling tissues. Although this indicates that HSI2 represses embryogenic programs in vegetative tissues, it has not been previously shown that HSI2 can positively contribute to seed oil accumulation. In fact, given that HSI2 represses embryogenesis, one would predict that overexpression would have a negative impact on seed oil accumulation. Thus, it is surprising that overexpression of HSI2 does lead to increases in seed oil accumulation. Since the nucleic acid molecule is a gene involved in regulating chromatin structure, HSI2 is likely to regulate seed oil accumulation by mechanisms that are distinct from those of previously reported genes.

### Terms

In order to facilitate review of the various embodiments of the disclosure, the following explanations of specific terms are provided:

Complementary nucleotide sequence: “Complementary nucleotide sequence” of a sequence is understood as meaning any DNA whose nucleotides are complementary to those of sequence of the disclosure, and whose orientation is reversed (antiparallel sequence).

Degree or percentage of sequence homology: The term “degree or percentage of sequence homology” refers to degree or percentage of sequence identity between two sequences after optimal alignment. Percentage of sequence identity (or degree or identity) is determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

Isolated: As will be appreciated by one of skill in the art, “isolated” refers to polypeptides or nucleic acids that have been “isolated” from their native environment.

Nucleotide, polynucleotide, or nucleic acid sequence: “Nucleotide, polynucleotide, or nucleic acid sequence” will be understood as meaning both a double-stranded or single-stranded DNA in the monomeric and dimeric (so-called in tandem) forms and the transcription products of said DNAs.

Sequence identity: Two amino-acid or nucleotide sequences are said to be “identical” if the sequence of amino-acids or nucleotidic residues in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman (Smith 1981), by the homology alignment algorithm of Neddleman and Wunsch (Neddleman 1970), by the search for similarity method of Pearson and Lipman (Pearson 1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Isolated and/or purified sequences of the present invention may have a percentage identity with the bases of a nucleotide sequence, or the amino acids of a polypeptide sequence, of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7%. This percentage is purely statistical, and it is possible to distribute the differences between the two nucleotide sequences at random and over the whole of their length.

The definition of sequence identity given above is the definition that would be used by one of skill in the art. The definition by itself does not need the help of any algorithm, said algorithms being helpful only to achieve the optimal alignments of sequences, rather than the calculation of sequence identity. From the definition given above, it follows that there is a well defined and only one value for the sequence identity between two compared sequences which value corresponds to the value obtained for the best or optimal alignment. In the BLAST N or BLAST P “BLAST 2 sequence”, software which is available in the web site http://www.ncbi.nlm.nih.gov/gorf/bl2.html, and habitually used by the inventors and in general by the skilled man for comparing and determining the identity between two sequences, gap cost which depends on the sequence length to be compared is directly selected by the software (i.e. 11.2 for substitution matrix BLOSUM-62 for length>85).

It will be appreciated that this disclosure embraces the degeneracy of codon usage as would be understood by one of ordinary skill in the art and as illustrated in Table 1. Furthermore, it will be understood by one skilled in the art that conservative substitutions may be made in the amino acid sequence of a polypeptide without disrupting the structure or function of the polypeptide. Conservative substitutions are accomplished by the skilled artisan by substituting amino acids with similar hydrophobicity, polarity, and R-chain length for one another. Additionally, by comparing aligned sequences of homologous proteins from different species, conservative substitutions may be identified by locating amino acid residues that have been mutated between species without altering the basic functions of the encoded proteins. Table 2 provides an exemplary list of conservative substitutions.

TABLE 1
Codon Degeneracies

| Amino Acid | Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, UGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, ACT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

TABLE 2
Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

### Expression

HSI2 nucleotide sequences can be expressed in alternate plant hosts to impart characteristics of improved agronomic performance via recombinant means. The methods to construct DNA expression vector and to transform and express foreign genes in plant and plant cells are well known in the art.

Additionally, it is evident that the sequences can be used in the construction of a construct or an expression vector. It is well known that nucleotide sequences encoding HSI2 can be inserted within an expression vector for heterologous expression in diverse host cells and organisms, for example plant cells and plant, by conventional techniques. These methods, which can be used in the invention, have been described elsewhere (Potrykus 1991; Vasil 1994; Walden 1995; Songstad 1995), and are well known to persons skilled in the art. As known in the art, there are a number of ways by which genes and gene constructs can be introduced into plants and a combination of transformation and tissue culture techniques have been successfully integrated into effective strategies for creating transgenic plants. For example, one skilled in the art will certainly be aware that, in addition to Agrobacterium-mediated transformation of Arabidopsis by vacuum infiltration (Bechtold 1993) or wound inoculation (Katavic 1994), it is equally possible to transform other plant species, using Agrobacterium Ti-plasmid mediated transformation (e.g., hypocotyl (DeBlock 1989) or cotyledonary petiole (Moloney 1989) wound infection), particle bombardment/biolistic methods (Sanford 1987; Nehra 1994; Becker 1994) or polyethylene glycol-assisted, protoplast transformation (Rhodes 1988; Shimamoto 1989) methods.

As will also be apparent to persons skilled in the art, and as described elsewhere (Meyer 1995; Datla 1997), it is possible to utilize plant promoters to direct any intended regulation of transgene expression using constitutive promoters (e.g., those based on CaMV35S), or by using promoters which can target gene expression to particular cells, tissues (e.g., napin promoter for expression of transgenes in developing seed cotyledons), organs (e.g., roots), to a particular developmental stage, or in response to a particular external stimulus (e.g., heat shock). Promoters for use herein may be inducible, constitutive, or tissue-specific or cell specific or have various combinations of such characteristics. Useful promoters include, but are not limited to constitutive promoters such as carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter). Meristem specific promoters include, for example, STM, BP, WUS, CLV gene promoters. Seed specific promoters include, for example, the napin promoter. Other cell and tissue specific promoters are well known in the art.

Promoter and termination regulatory regions that will be functional in the host plant cell may be heterologous (that is, not naturally occurring) or homologous (derived from the plant host species) to the plant cell and the gene. Suitable promoters which may be used are described above. The termination regulatory region may be derived from the 3′ region of the gene from which the promoter was obtained or from another gene. Suitable termination regions which may be used are well known in the art and include Agrobacterium tumefaciens nopaline synthase terminator (Tnos), A. tumefaciens mannopine synthase terminator (Tmas) and the CaMV 35S terminator (T35S). Particularly preferred termination regions for use herein include the pea ribulose bisphosphate carboxylase small subunit termination region (TrbcS) or the Tnos termination region. Such gene constructs may suitably be screened for activity by transformation into a host plant via Agrobacterium and screening for the desired activity using known techniques.

Preferably, a nucleic acid molecule construct for use herein is comprised within a vector, most suitably an expression vector adapted for expression in an appropriate plant cell. It will be appreciated that any vector which is capable of producing a plant comprising the introduced nucleic acid sequence will be sufficient. Suitable vectors are well known to those skilled in the art and are described in general technical references. Particularly suitable vectors include the Ti plasmid vectors. After transformation of the plant cells or plant, those plant cells or plants into which the desired nucleic acid molecule has been incorporated may be selected by such methods as antibiotic resistance, herbicide resistance, tolerance to amino-acid analogues or using phenotypic markers. Various assays may be used to determine whether the plant cell shows an increase in gene expression, for example, Northern blotting or quantitative reverse transcriptase PCR (RT-PCR). Whole transgenic plants may be regenerated from the transformed cell by conventional methods. Such plants produce seeds containing the genes for the introduced trait and can be grown to produce plants that will produce the selected phenotype.

Expression or overexpression of genes encoding HSI2 may done in combination with overexpression or expression of one or more other genes involved in seed oil production and/or abiotic stress tolerance, for example, lipid biosynthetic genes such as diacylglycerol acyltransferase (DGAT) or genes encoding regulatory elements including kinases such as pyruvate dehydrogenase complex kinase (PDCK) and transcription factors such as WRINKLED1 (WRI1).

Preferred plants in which HSI2 activity may be expressed or overexpressed include crop species, especially oilseed plant species. Some examples include Brassicaceae spp. (e.g. rapeseed and Canola), Borago spp. (borage), Ricinus spp. (e.g. Ricinus communis (castor)), Theobroma spp. (e.g. Theobroma cacao (cocoa bean)), Gossypium spp. (cotton), Crambe spp., Cuphea spp., Linum spp. (flax), Lesquerella spp., Limnanthes spp., Linola, Tropaeolum spp. (nasturtium), Olea spp. (olive), Elaeis spp. (palm), Arachis spp. (peanut), Carthamus spp. (safflower), Glycine spp. (soybean), Soja spp. (soybean), Helianthus spp. (sunflower), Vemonia spp. Plants of particular note are from the family Brassicaceae, especially Arabidopsis thaliana, Brassica napus, Brassica rapa, Brassica carinata, Brassica juncea, and Camelina sativa. Arabidopsis thaliana, Brassica spp. and Glycine spp. are of particular note.

### Silencing

Silencing of HSI2 genes may be accomplished in a number of ways generally known in the art, for example, RNA interference (RNAi) techniques, artificial microRNA techniques, virus-induced gene silencing (VIGS) techniques, antisense techniques, sense co-suppression techniques and targeted mutagenesis techniques.

RNAi techniques involve stable transformation using RNA interference (RNAi) plasmid constructs (Helliwell 2005). Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant and cut into small RNAs (about 21 nucleotides long) called small interfering RNAs (siRNAs). siRNAs associate with a protein complex (RISC) which goes on to direct degradation of the mRNA for the target gene.

Artificial microRNA (amiRNA) techniques exploit the microRNA (miRNA) pathway that functions to silence endogenous genes in plants and other eukaryotes (Schwab 2006; Alvarez 2006). In this method, 21 nucleotide long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes which share nucleotide identity with the 21 nucleotide amiRNA sequence.

In RNAi silencing techniques, two factors can influence the choice of length of the fragment. The shorter the fragment the less frequently effective silencing will be achieved, but very long hairpins increase the chance of recombination in bacterial host strains. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences. Many of these same principles apply to selection of target regions for designing amiRNAs.

Virus-induced gene silencing (VIGS) techniques are a variation of RNAi techniques that exploits the endogenous antiviral defenses of plants. Infection of plants with recombinant VIGS viruses containing fragments of host DNA leads to post-transcriptional gene silencing for the target gene. In one embodiment, a tobacco rattle virus (TRV) based VIGS system can be used.

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Application of antisense to gene silencing in plants is described in more detail by Stam 2000.

Sense co-suppression techniques involve introducing a highly expressed sense transgene into a plant resulting in reduced expression of both the transgene and the endogenous gene (Depicker 1997). The effect depends on sequence identity between transgene and endogenous gene.

Targeted mutagenesis techniques, for example TILLING (Targeting Induced Local Lesions IN Genomes) and “delete-a-gene” using fast-neutron bombardment, may be used to knockout gene function in a plant (Henikoff 2004; Li 2001). TILLING involves treating seeds or individual cells with a mutagen to cause point mutations that are then discovered in genes of interest using a sensitive method for single-nucleotide mutation detection. Detection of desired mutations (e.g. mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e. deletions, in plant genomes that can also be detected using PCR in a manner similar to TILLING.

### EXAMPLES

### Example 1

### Transformation of Plant Material

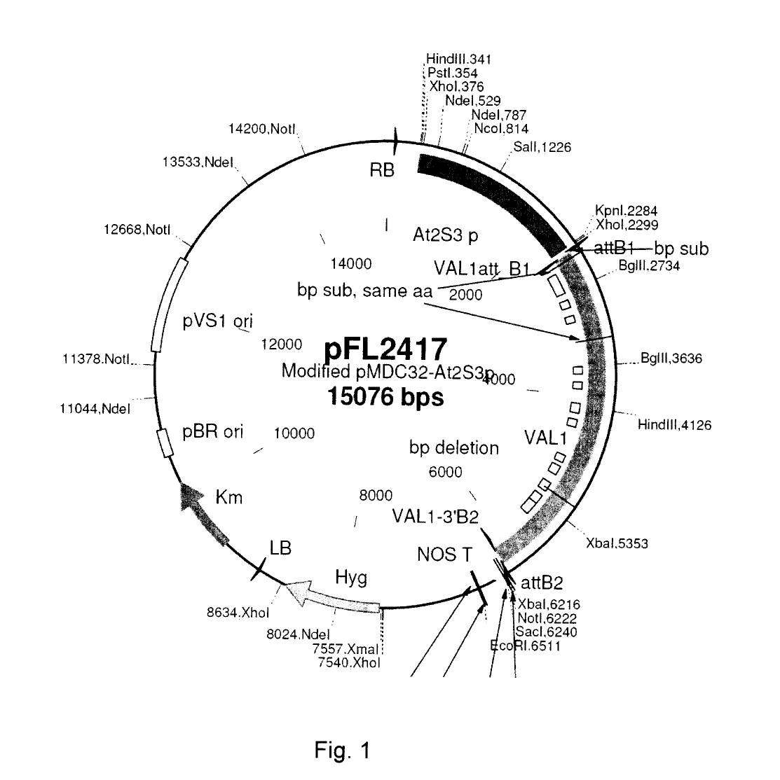

The genomic protein coding region of At2g30470, was cloned from the bacterial artificial chromosome T6B20 into cloning vector pJM1 by recombination as described by Liu et al. (Liu 2003) and subsequently into the binary T-DNA vectors pDMC32:At2S3 and pER330 using Gateway technology (Invitrogen). Agrobacterium strain GV3101 (MP90) harboring the T-DNA vector was used to transform the At2S3 (napin):HSI2 and cauliflower mosaic virus 35S:HSI2 gene constructs into Arabidopsis thaliana (Columbia-0 ecotype) by floral dipping. The vectors were also transformed into Brassica napus (DH12075) as described by Zou et al. (Zou 1997). The At2S3 (napin):HSI2 vector map is shown in FIG. 1.

Two putative hsi2 T-DNA insertion lines were identified in Arabidopsis thaliana (Columbia-0 and Columbia-2 backgrounds) from the Salk Institute Genomic Laboratory Genomic database (http://signal.salk.edu) and seeds were obtained from the Arabidopsis Biological Resource Centre (ABRC). Homozygote T-DNA insertion lines were identified though PCR genotyping. hsi2-5 (Salk_{—}088606) was identified in Columbia-0 background and hsi2-3 (WiscDsLox388F10) was identified in Columbia-2 background. The insertion lines were analyzed for seed oil content, fatty acid profile, and ABA responses during germination and drought tolerance in juvenile plants.

### Example 2

### Seed Oil Analysis

Seeds of T-DNA insertion mutant lines of HSI2 and the wild type were stratified in the dark for 3 days at 4° C. and sown on Sunshine™ Mix4 germination medium (Sun Gro Horticulture, Canada). Plants were germinated and grown in a growth chamber (Conviron) under 16-hr photoperiod, 21/18° C. day/night temperature cycle and about 250 μE light intensity. Secondary shoots were trimmed out and siliques on primary shoots were allowed to dry on plants before moving to a finishing chamber for another 2 weeks to ensure complete ripening of seeds. Seeds were harvested only from main shoots and allowed to dry at room temperature for 2-3 weeks before analyzing for seed oil content and fatty acid profiles.

Total lipids were extracted by grinding seeds in chloroform:isopropanol (2:1). The solvent was evaporated off at room temperature under a stream of nitrogen gas and total lipids were transmethylated by heating samples with 3 N methanolic HCl at 80° C. for 3 hrs. Fatty acids methyl esters (FAMES) were then extracted with GC grade hexane in presence of 0.9% NaCl. The solvent (hexane) was evaporated under a stream of nitrogen gas and FAMES were re-dissolved in 500 μl of methyl esters standards (17:0 M.E. and 23:0 M.E.) in hexane and analyzed by GC. Seed oil content and fatty acid profiles were calculated as a percentage (%) of dry seed weight.

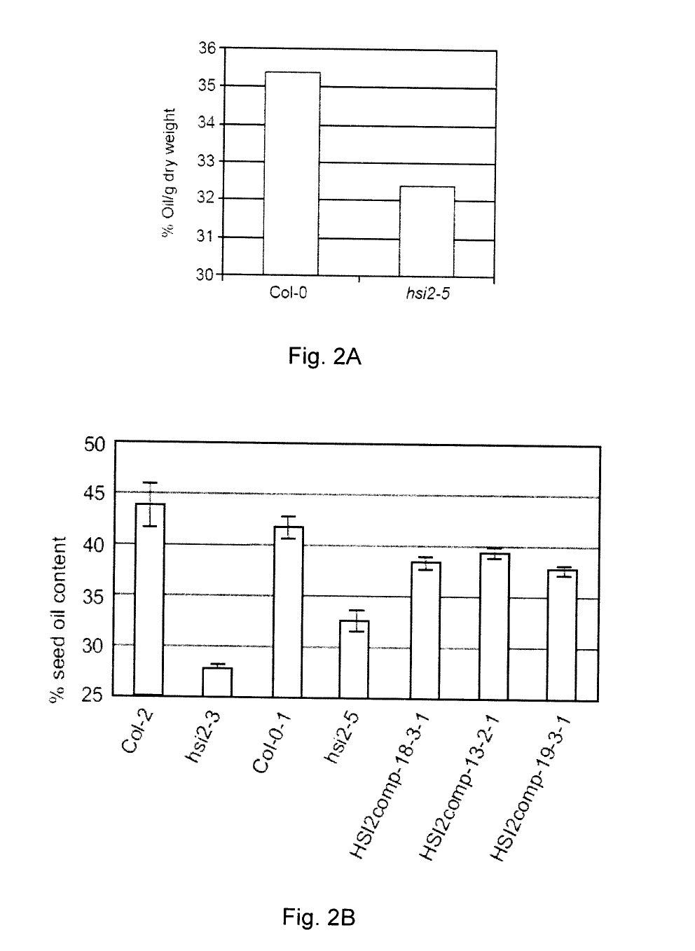

Oil content was measured on the Arabidopsis hsi2-5 T-DNA insertion mutant lines along with its wild type at four separate times, and the mutant shows a decrease in seed oil content (FIG. 2A). However, profiles of fatty acids were not altered in the mutant line. On the other hand, preliminary oil analysis on primary transformants in Brassica napus (DH12075) over expressing HSI2 in seeds under the control of napin and 35S promoters indicated a slight increase in seed oil content (data not shown).

Comparing wild type (Col-2 and Col-0-1) to T-DNA insertion mutant lines (hsi2-3 and hsi2-5) and three HSI2 complementation lines (complementation in hsi2-5; HSI2 comp-18-3-1, HSI2 comp-12-2-1 and HSI2 comp-19-3-1) (FIG. 2B), it can be seen that the mutants have less oil than their corresponding wild types suggesting that HSI2 gene may positively regulate seed oil content. Specificity of the mutation is shown by complementation of seed oil content of hsi2-5. The hsi2-5 mutant was transformed with the HSI2 gene under its own promoter.

### Example 3

### Germination

Seeds were surface-sterilized with 30% bleach (0.01% Tween™-20), rinsed several times with sterilized water, and sown on 1.5% agar plates containing half-strength Murashige and Skoog (MS) salt solution supplemented or not with 0 μM, 0.25 μM or 0.3 μM ABA (±ABA, Toray batch; PBI 58, NRC/PBI Saskatoon). The plates were transferred to a tissue culture room after a cold treatment of 2 days at 4° C. in the dark, and incubated at 20° C./16° C. day/night temperatures under a 16 hrs/8 hrs light/dark regime and 80-100 μE irradiance.

Germination (defined as endosperm rupture and radical emergence) was scored starting 24 hrs after seed stratification. Germination events are expressed as a percentage of the total number of seeds per plate. Germination experiments were repeated at least three times using three different seed batches of wild type and T-DNA insertion mutant lines (hsi2-5 and hsi2-3) grown in parallel.

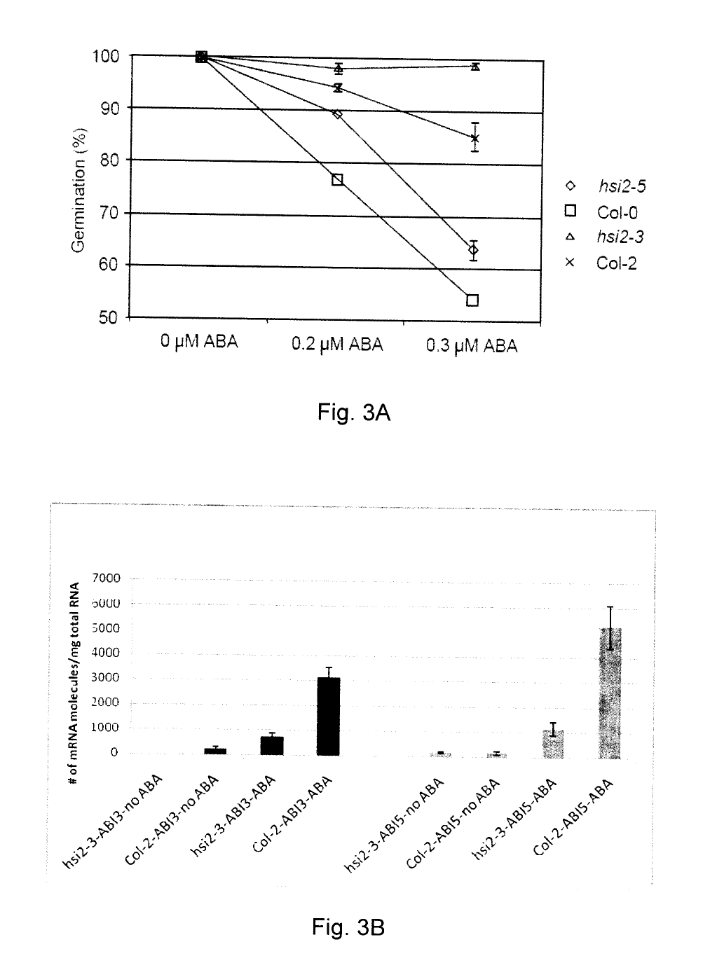

Both of the T-DNA insertion mutant liens showed a decreased sensitivity to ABA during germination as indicated by faster and higher overall germination than in the corresponding wild type in presence of ABA in the germination medium (FIG. 3A).

ABI3 and ABI5 are two genes known to inhibit seed germination by arresting embryo growth. Both hsi2 alleles show minimal or no expression of these genes during seed germination 96 hr after stratification (FIG. 3B). This could explain why these mutants germinate better in presence of ABA.

### Example 4

### Drought Responses

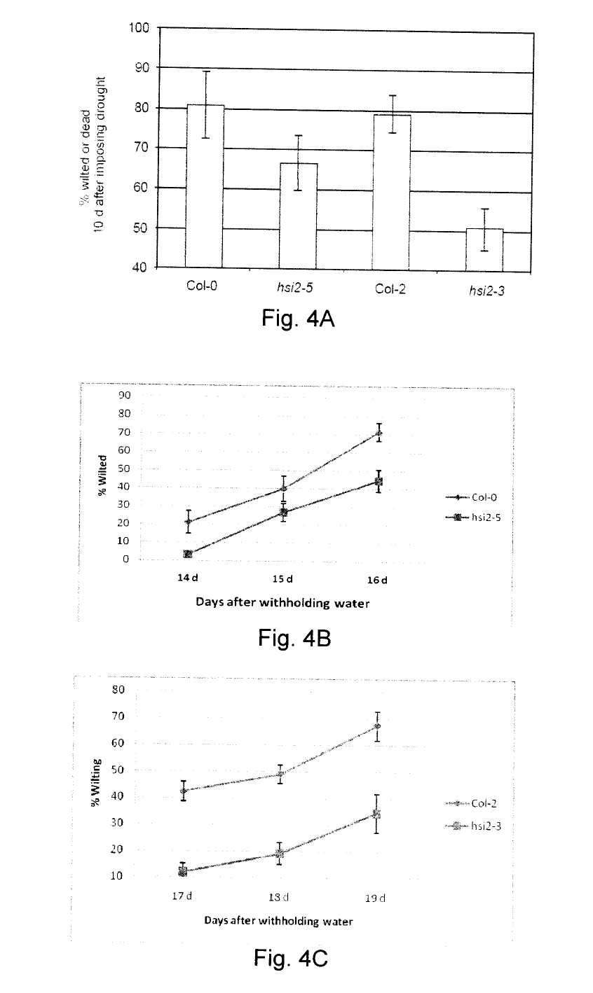

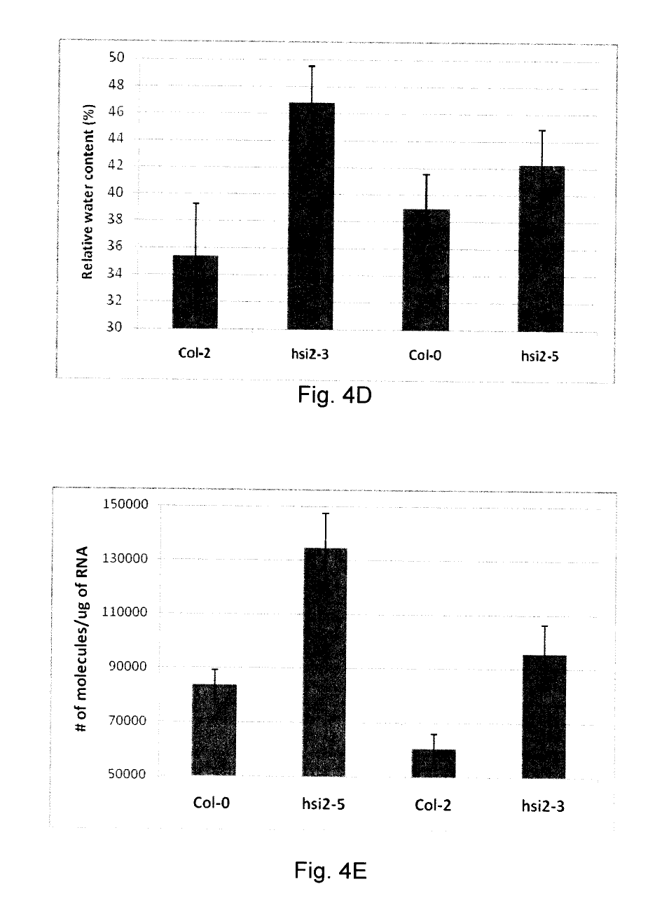

Wild type and the mutant seeds were germinated directly on Sunshine™ Mix4 (Sun Gro Horticulture, Canada) and grown under regular watering and fertilization regime until the plants were three-weeks-old. At that point, plants were subjected to drought stress by withholding water and wilting symptoms were monitored daily thereafter until more than 80% plants displayed some degree of wilting. Drought response was evaluated in three independent batches of plants. Results are presented as a percentage of wilted or dead plants 10 days (10 d) after imposing drought stress by withholding water. Both of the hsi2 T-DNA insertion mutant lines showed a reduced sensitivity to drought as indicated by lower percentage wilting or death of the whole plant compared to their wild types at a certain time point after imposing drought stress (FIG. 4A). The same pattern continued from 14-16 days without water for hsi2-5 (FIG. 4B) and from 17-19 days without water for hsi2-3 (FIG. 4C).

Leaf relative water content at 19 days of withholding water was also determined for his2 mutants compared to their wild type (FIG. 4D). Leaf relative water content is an indicator of how well a plant is able to maintain its water status during a stress. The his2 mutants maintain higher leaf relative water content during drought stress than their wild types.

Myb-like transcription factor is one of the genes involved in regulating stomatal aperture and hence potentially involved in plant water use. Expression of Myb-like transcription factor gene in hsi2 mutant plants approaching visible wilting was determined. hsi2 mutants express higher levels of this gene in their leaves (FIG. 4E) than their wild types, which may help in regulating water loss through transpiration and hence more drought tolerance.

Free Listing of Sequences

HSI2-nucleotide sequence (SEQ ID NO: 1)-Arabidopsis thaliana (2373 bp)
ATGTTTGAAGTCAAAATGGGGTCAAAGATGTGCATGAACGCTTCATGTGGTACGACTTCTACTGTT
GAATGGAAGAAAGGTTGGCCTCTTCGATCTGGTCTTCTCGCTGATCTCTGTTATCGTTGCGGATCT
GCGTATGAGAGTTCTCTATTCTGTGAACAATTTCATAAGGACCAATCTGGTTGGAGGGAATGCTAT
TTGTGTAGCAAGAGACTACATTGTGGATGCATTGCTTCTAAGGTAACGATTGAGTTAATGGACTAT
GGTGGTGTTGGTTGTAGTACATGTGCTTGCTGCCATCAACTCAATTTGAACACAAGGGGTGAGAAT
CCAGGTGTTTTTAGCAGATTGCCAATGAAAACGTTAGCTGATAGGCAACATGTAAATGGCGAAAGC
GGAGGAAGAAACGAAGGCGATCTCTTTTCTCAGCCACTAGTCATGGGCGGAGATAAAAGGGAAGAG
TTCATGCCTCACCGTGGGTTTGGTAAGCTAATGAGTCCAGAAAGTACAACCACCGGGCATAGGCTG
GATGCTGCTGGGGAAATGCATGAATCATCACCTTTACAGCCATCTTTAAATATGGGTTTGGCTGTG
AATCCGTTTAGCCCATCTTTTGCAACCGAGGCTGTCGAGGGAATGAAACACATCAGTCCTTCTCAG
TCCAACATGGTCCATTGCTCTGCTTCTAATATACTGCAAAAGCCATCAAGACCTGCTATTTCAACT
CCTCCTGTGGCTAGTAAATCCGCTCAGGCGCGGATTGGAAGGCCTCCTGTCGAAGGGCGAGGGAGA
GGCCACTTGCTTCCGCGGTATTGGCCAAAATATACGGATAAAGAGGTTCAGCAGATCTCTGGAAAT
TTGAATTTGAACATTGTACCTCTCTTTGAGAAAACTCTTAGTGCCAGTGATGCTGGTCGCATTGGT
CGTCTAGTTCTTCCAAAAGCCTGTGCAGAGGCATATTTTCCTCCGATTAGTCAATCCGAAGGCATT
CCTTTGAAAATCCAAGATGTGAGGGGTAGGGAGTGGACGTTCCAGTTCAGATATTGGCCCAATAAC
AATAGTAGAATGTATGTTTTAGAAGGTGTCACTCCATGCATACAGTCCATGATGCTACAGGCTGGT
GATACAGTAACTTTCAGTCGGGTTGATCCTGGCGGAAAACTAATCATGGGTTCCAGGAAGGCAGCT
AATGCTGGAGACATGCAGGGTTGTGGGCTCACCAACGGAACATCAACTGAGGACACATCATCGTCT
GGTGTAACAGAAAACCCACCCTCCATAAATGGTTCCTCGTGTATTTCACTAATACCGAAAGAGTTG
AATGGTATGCCTGAGAATTTGAACAGTGAGACTAACGGGGGCAGGATAGGTGATGATCCTACACGA
GTTAAAGAGAAGAAGAGAACTCGAACCATTGGTGCAAAAAATAAGAGACTTCTTTTGCATAGTGAA
GAATCTATGGAGCTGAGACTCACTTGGGAAGAAGCTCAGGACTTGCTTCGTCCCTCTCCTAGTGTA
AAGCCTACCATCGTTGTCATTGAGGAGCAAGAAATTGAAGAATATGACGAACCTCCTGTCTTTGGA
AAGAGGACTATAGTCACTACAAAACCTTCAGGTGAACAGGAACGATGGGCAACTTGCGACGACTGC
TCTAAATGGAGAAGGTTACCTGTAGATGCTCTTCTTTCCTTTAAATGGACATGTATAGACAATGTT
TGGGATGTGAGTAGGTGTTCATGTTCTGCACCGGAGGAGAGTCTGAAGGAACTTGAGAATGTTCTT
AAAGTAGGAAGAGAGCACAAGAAGAGAAGAACTGGGGAAAGCCAGGCAGCAAAAAGTCAGCAAGAA
CCGTGTGGTTTGGACGCACTGGCGAGTGCAGCAGTCTTAGGAGACACAATAGGCGAGCCAGAGGTA
GCGACCACGACCAGACATCCAAGGCACAGGGCTGGATGCTCTTGCATCGTGTGCATTCAGCCACCA
AGTGGGAAAGGTAGGCACAAGCCTACATGTGGCTGCACTGTGTGTAGCACCGTGAAGAGAAGGTTC
AAGACGCTTATGATGAGGAGGAAGAAGAAGCAGTTGGAGCGCGATGTAACAGCAGCAGAAGATAAG
AAGAAGAAGGACATGGAACTGGCTGAGTCTGATAAGAGTAAGGAGGAGAAGGAAGTGAACACAGCG
AGAATAGACCTGAACAGTGATCCATACAATAAAGAAGATGTTGAAGCTGTTGCGGTGGAGAAAGAA
GAGAGTCGAAAAAGAGCAATAGGACAGTGTTCGGGCGTGGTGGCTCAAGACGCCAGTGATGTTTTA
GGAGTTACAGAGTTAGAAGGAGAGGGTAAGAATGTTCGTGAAGAGCCGAGAGTTTCAAGCTGA
HSI2-amino acid sequence (SEQ ID NO: 2)-Arabidopsis thaliana (790 aa)
MFEVKMGSKMCMNASCGTTSTVEWKKGWPLRSGLLADLCYRCGSAYESSLFCEQFHKDQSGWRECY
LCSKRLHCGCIASKVTIELMDYGGVGCSTCACCHQLNLNTRGENPGVFSRLPMKTLADRQHVNGES
GGRNEGDLFSQPLVMGGDKREEFMPHRGFGKLMSPESTTTGHRLDAAGEMHESSPLQPSLNMGLAV
NPFSPSFATEAVEGMKHISPSQSNMVHCSASNILQKPSRPAISTPPVASKSAQARIGRPPVEGRGR
GHLLPRYWPKYTDKEVQQISGNLNLNIVPLFEKTLSASDAGRIGRLVLPKACAEAYFPPISQSEGI
PLKIQDVRGREWTFQFRYWPNNNSRMYVLEGVTPCIQSMMLQAGDTVTFSRVDPGGKLIMGSRKAA
NAGDMQGCGLTNGTSTEDTSSSGVTENPPSINGSSCISLIPKELNGMPENLNSETNGGRIGDDPTR
VKEKKRTRTIGAKNKRLLLHSEESMELRLTWEEAQDLLRPSPSVKPTIVVIEEQEIEEYDEPPVFG
KRTIVTTKPSGEQERWATCDDCSKWRRLPVDALLSFKWTCIDNVWDVSRCSCSAPEESLKELENVL
KVGREHKKRRTGESQAAKSQQEPCGLDALASAAVLGDTIGEPEVATTTRHPRHRAGCSCIVCIQPP
SGKGRHKPTCGCTVCSTVKRRFKTLMMRRKKKQLERDVTAAEDKKKKDMELAESDKSKEEKEVNTA
RIDLNSDPYNKEDVEAVAVEKEESRKRAIGQCSGVVAQDASDVLGVTELEGEGKNVREEPRVSS

References: The contents of the entirety of each of which are incorporated by this reference.
GenBank Accession No. NM_{—}179815, GI 30684597, AT2G30470 (2008).
Alvarez J P, Pekker I, Goldshmidt A, Blum E, Amsellem Z, Eshed Y. (2006) Endogenous and synthetic microRNAs stimulate simultaneous, efficient, and localized regulation of multiple targets in diverse species. Plant Cell. 8: 1134-51.
Baud S, Mendoza M S, To A, Harscoet E, Lepiniec L, Dubreucq B. (2007) WRINKLED1 specifies the regulatory action of LEAFY COTYLEDON2 towards fatty acid metabolism during seed maturation in Arabidopsis. Plant J. 50: 825-828.
Bechtold N, Ellis J, Pellefer G. (1993) In planta Agrobacterium-mediated gene transfer by infiltration of adult Arabidopsis thaliana plants. C.R. Acad. Sci. Ser. III Sci. Vie, 316: 1194-1199.
Becker D, Brettschneider R, Lorz H. (1994) Fertile transgenic wheat from microprojectile bombardment of scutellar tissue. Plant J. 5: 299-307.
Datla R, Anderson J W, Selvaraj G. (1997) Plant promoters for transgene expression. Biotechnology Annual Review. 3: 269-296.
DeBlock M, DeBrouwer D, Tenning P. (1989) Transformation of Brassica napus and Brassica oleracea using Agrobacterium tumefaciens and the expression of the bar and neo genes in the transgenic plants. Plant Physiol. 91: 694-701.
Depicker A, Montagu M V. (1997) Post-transcriptional gene silencing in plants. Curr Opin Cell Biol. 9: 373-82.
Gutierrez L, Van Wuytswinkel O, Castelain M, Bellini C. (2007) Combined networks regulating seed maturation. Trends Plant Sci. 12: 294-300.
Helliwell C A, Waterhouse P M. (2005) Constructs and methods for hairpin RNA-mediated gene silencing in plants. Methods Enzymology 392: 24-35.
Henikoff S, Till B J, Comai L. (2004) TILLING. Traditional mutagenesis meets functional genomics. Plant Physiol. 135: 630-6.
Jako C, Kumar A, Wei Y, Zou J, Barton D L, Giblin E M, Covello P S, Taylor D C. (2001) Seed-specific over-expression of an Arabidopsis cDNA encoding a diacylglycerol acyltransferase enhances seed oil content and seed weight. Plant Physiol. 126: 861-874.
Katavic Y, Haughn G W, Reed D, Martin M, Kunst L. (1994) In planta transformation of Arabidopsis thaliana. Mol Gen. Genet. 245: 363-370.
Li X, Song Y, Century K, Straight S, Ronald P, Dong X, Lassner M, Zhang Y. (2001) A fast neutron deletion mutagenesis-based reverse genetics system for plants. Plant J. 27: 235-242.
Liu P, Jenkins N A, Copeland N G. (2003) A Highly Efficient Recombineering-Based Method for Generating Conditional Knockout Mutations. Genome Res. 13 (3): 476-484.
Meyer P. (1995) Understanding and controlling transgene expression. Trends in Biotechnology. 13: 332-337.
Moloney M M, Walker J M, Sharma K K. (1989) High efficiency transformation of Brassica napus using Agrobacterium vectors. Plant Cell Rep. 8: 238-242.
Neddleman and Wunsch. (1970) J. Mol. Biol. 48: 443.
Nehra N S, Chibbar R N, Leung N, Caswell K, Mallard C, Steinhauer L, Baga M, Kartha K K. (1994) Self-fertile transgenic wheat plants regenerated from isolated scutellar tissues following microprojectile bombardment with two distinct gene constructs. Plant J. 5: 285-297.
Pearson and Lipman. (1988) Proc. Natl. Acad. Sci. (U.S.A.) 85: 2444.
Potrykus L. (1991) Gene transfer to plants: Assessment of publish approaches and results. Annu. Rev. Plant Physiol. Plant Mol. Biol. 42: 205-225.
Rhodes C A, Pierce D A, Mettler I J, Mascarenhas D, Detmer J J. (1988) Genetically transformed maize plants from protoplasts. Science. 240: 204-207.
Sambrook J, Fritsch E F, Maniatis T. (1989) Molecular Cloning: A Laboratory Manual 2^{nd} edn. Cold Spring Harbor: Cold Spring Harbor Laboratory Press.
Sambrook J, Fritsch E F, Maniatis T. (2001) Molecular Cloning: A Laboratory Manual 3^{rd} edn. Cold Spring Harbor: Cold Spring Harbor Laboratory Press.
Santos-Mendoza M, Dubreucq B, Baud S, Parcy F, Caboche M, Lepiniec L. (2008) Deciphering gene regulatory networks that control seed development and maturation in Arabidopsis. Plant J. 54: 608-620.
Schwab R, Ossowski S, Riester M, Warthmann N, Weigel D. (2006) Highly specific gene silencing by artificial microRNAs in Arabidopsis. Plant Cell. 18: 1121-33.
Shimamoto K, Terada R, Izawa T, Fujimoto H. (1989) Fertile transgenic rice plants regenerated from transformed protoplasts. Nature. 335: 274-276.
Smith and Waterman. (1981) Ad. App. Math. 2: 482.
Songstad D D, Somers D A, Griesbach R J. (1995) Advances in alternative DNA delivery techniques. Plant Cell, Tissue and Organ Culture. 40: 1-15.
Stam M, de Bruin R, van Blokland R, van der Hoorn R A, Mol J N, Kooter J M. (2000) Distinct features of post-transcriptional gene silencing by antisense transgenes in single copy and inverted T-DNA repeat loci. Plant J. 21: 27-42.
Suzuki M, Wang H H, McCarty D R. (2007) Repression of the LEAFY COTYLEDON 1/B3 regulatory network in plant embryo development by VP1/ABSCISIC ACID INSENSITIVE 3-LIKE B3 genes. Plant Physiol. 143(2): 902-11.
Taylor D C, Zhang Y, Kumar A, Francis T, Giblin E M, Barton D L, Ferrie J R, Laroche A, Shah S, Zhu W, et al. (2008) Molecular modification of triacylglycerol accumulation under field conditions to produce canola with increased seed oil content. Botany. In press.
Tsukagoshi H, Saijo T, Shibata D, Morikami A, Nakamura K. (2005) Analysis of a sugar response mutant of Arabidopsis identified a novel B3 domain protein that functions as an active transcriptional repressor. Plant Physiol. 138(2): 902-911.
Tsukagoshi H, Morikami A, Nakamura K. (2007) Two B3 domain transcriptional repressors prevent sugar-inducible expression of seed maturation genes in Arabidopsis seedlings. PNAS. 104(7): 2543-7.
Vasil I K. (1994) Molecular improvement of cereals. Plant Mol. Biol. 5: 925-937.
Vigeolas H, Waldeck P, Zank T, Geigenberger P. (2007) Increasing seed oil content in oil-seed rape (Brassica napus L.) by over-expression of a yeast glycerol-3-phosphate dehydrogenase under the control of a seed-specific promoter. Plant Biotechnol J. 5: 431-441.
Walden R, Wingender R. (1995) Gene-transfer and plant regeneration techniques. Trends in Biotechnology. 13: 324-331.
Wang H W, Zhang B, Hao Y J, Huang J, Tian A G, Liao Y, Zhang J S, Chen S Y. (2007) The soybean Dof-type transcription factor genes, GmDof4 and GmDof11, enhance lipid content in the seeds of transgenic Arabidopsis plants. Plant J. 52: 716-729.
Zou J, Katavic V, Giblin E M, Barton D L, MacKenzie S L, Keller W A, Hu X, Taylor D C. (1997) Modification of seed oil content and acyl composition in the brassicaceae by expression of a yeast sn-2 acyltransferase gene. Plant Cell. 9: 909-923.
Zou J, Qi Q, Katavic V, Marillia E F, Taylor D C. (1999) Effects of antisense repression of an Arabidopsis thaliana pyruvate dehydrogenase kinase cDNA on plant development. Plant Mol Biol. 41: 837-849.
Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of decreasing abscisic acid sensitivity and/or increasing drought resistance in a plant comprising: reducing expression of HIGH-LEVEL EXPRESSION OF SUGAR-INDUCIBLE 2 (HSI2) protein in the plant to thereby decrease abscisic acid sensitivity and/or increase drought resistance in the plant compared to a plant grown under similar conditions in which expression of the HSI2 protein has not been reduced.

2. A method of increasing seed oil content in a plant comprising: introducing into the plant means for encoding a HIGH-LEVEL EXPRESSION OF SUGAR-INDUCIBLE 2 (HSI2) protein to thereby increase expression of HSI2 protein in the plant to thereby increase seed oil content in the plant compared to a plant grown under similar conditions in which the means for encoding the HSI2 protein has not been introduced.

3. The method according to claim 2, wherein the means for encoding comprises a nucleic acid molecule having a nucleotide sequence having at least 80% sequence identity to SEQ ID NO: 1 or a codon degenerate nucleotide sequence thereof.

4. The method according to claim 3, wherein the nucleic acid molecule has a nucleotide sequence as set forth in SEQ ID NO: 1 or a codon degenerate nucleotide sequence thereof.

5. The method according to claim 1, wherein the HSI2 protein has an amino acid sequence having at least 80% sequence identity to SEQ ID NO: 2 or a conservatively substituted amino acid sequence thereof.

6. The method according to claim 1, wherein the HSI2 protein has an amino acid sequence as set forth in SEQ ID NO: 2 or a conservatively substituted amino acid sequence thereof.

7. The method according to claim 1, wherein the plant is from family Brassicaceae.

8. A nucleic acid construct comprising means for encoding a HIGH-LEVEL EXPRESSION OF SUGAR-INDUCIBLE 2 (HSI2) protein operably linked to one or more nucleic acid sequences required for transforming the construct into a cell and/or for expressing or overexpressing the HSI2 protein encoding means in the cell.

9. The construct according to claim 8, wherein the HSI2 protein has an amino acid sequence having at least 80% sequence identity to SEQ ID NO: 2 or a conservatively substituted amino acid sequence thereof.

10. The construct according to claim 8, wherein the HSI2 protein has an amino acid sequence as set forth in SEQ ID NO: 2 or a conservatively substituted amino acid sequence thereof.

11. A cell, seed or plant comprising the nucleic acid construct as defined in claim 8.

12. The cell, seed or plant according to claim 11, wherein the HSI2 protein has an amino acid sequence having at least 80% sequence identity to SEQ ID NO: 2 or a conservatively substituted amino acid sequence thereof.

13. The cell, seed or plant according to claim 11, wherein the HSI2 protein has an amino acid sequence as set forth in SEQ ID NO: 2 or a conservatively substituted amino acid sequence thereof.

14. The method according to claim 2, wherein the HSI2 protein has an amino acid sequence having at least 80% sequence identity to SEQ ID NO: 2 or a conservatively substituted amino acid sequence thereof.

15. The method according to claim 2, wherein the HSI2 protein has an amino acid sequence as set forth in SEQ ID NO: 2 or a conservatively substituted amino acid sequence thereof.

16. The method according to claim 2, wherein the plant is from family Brassicaceae.

17. The method according to claim 3, wherein the HSI2 protein has an amino acid sequence having at least 80% sequence identity to SEQ ID NO: 2 or a conservatively substituted amino acid sequence thereof.

18. The method according to claim 3, wherein the HSI2 protein has an amino acid sequence as set forth in SEQ ID NO: 2 or a conservatively substituted amino acid sequence thereof.

19. The method according to claim 3, wherein the plant is from family Brassicaceae.

20. The method according to claim 4, wherein the HSI2 protein has an amino acid sequence having at least 80% sequence identity to SEQ ID NO: 2 or a conservatively substituted amino acid sequence thereof.

21. The method according to claim 4, wherein the HSI2 protein has an amino acid sequence as set forth in SEQ ID NO: 2 or a conservatively substituted amino acid sequence thereof.

22. The method according claim 4, wherein the plant is from family Brassicaceae.
